(19) United States
(12) Patent Application Publication
(10) Pub. No.: US 2013/0197425 A1
(43) Pub. Date: Aug. 1, 2013

(54) CURRENT CAGE FOR REDUCTION OF A NON-TARGET TISSUE EXPOSURE TO ELECTRIC FIELDS IN ELECTROPORATION BASED TREATMENT

(71) Applicant: The Regents Of The University Of California, Oakland, CA (US)

(72) Inventors: Alex GOLBERG, Jerusalem (IL); Boris RUBINSKY, El Cerrito, CA (US)

(73) Assignee: THE REGENTS OF THE UNIVERSITY OF CALIFORNIA, Oakland, CA (US)

(21) Appl. No.: 13/714,862

(22) Filed: Dec. 14, 2012

Related U.S. Application Data
(60) Provisional application No. 61/579,208, filed on Dec. 22, 2011.
(60) Provisional application No. 61/576,865, filed on Dec. 16, 2011.

(51) Int. Cl. A61N 1/32; A61B 18/14

(52) U.S. Cl. CPC A61N 1/325; A61B 18/14
USPC 604/20; 606/41

(57) ABSTRACT

The invention shows the relation between the volumes of tissue that experiences muscle contraction inducing electric fields, V_{MC} for various electroporation volumes, V_{E} and electroporation electrodes design. The inductive electric fields, produced by the transient changes in current flow in electroporation electrodes, are not sufficient to induce muscle contractions. However, the direct current delivered by electrodes can produce substantial volumes of muscle contraction inducing electric fields. Electrode placements are designed in such a way as to substantially reduce the volume of V_{MC} for the same V_{E}. Employing electrodes in a structure referred to as a Current Cage reduces substantially the V_{MC} relative to a standard two electrode needle system.

### CROSS-REFERENCE

This application claims the benefit of U.S. Provisional Application Nos. 61/576,865, filed Dec. 16, 2001 and 61/579,208, filed Dec. 22, 2011, which applications are incorporated herein by reference.

### FIELD OF THE INVENTION

The invention relates to the field of electroporation and specifically to electrode shapes and configurations used to reduce the volume of the non-target tissue exposure to the electric fields.

### BACKGROUND OF THE INVENTION

When certain electrical fields are applied across a cell, they have the ability to permeabilize the cell membrane, presumably through the formation of nanoscale defects—pores—in the membrane. The process of cell membrane permeabilization by pulsed electric fields (PEF) was coined “electroporation” in the early 80's (Neumann, Schaefer-Ridder, Wang, & Hofschneider, 1982). Dev et al mapped the fate of cells as a function of applied pulse field strength and duration showing that under the application of specific electric fields the electroporation phenomena may be reversible and cell survive; while for longer pulses and higher fields the effect is irreversible and the cell dies (Dev, Rabussay, Widra, & Hofmann, 2000).

Reversible electroporation has become an important tool in biotechnology and medicine (L. M. Mir, 2001). It facilitates the introduction of otherwise non-permeable external substances into cells while keeping cells alive. Applications of reversible electroporation include gene delivery to cells (Neumann et al., 1982) and tissues (Titomirov, Sukharev, & Kistanova, 1991), and the introduction of drugs into cells (Okino & Mohri, 1987). Reversible electroporation is the basis for a successful cancer treatment therapy known as “electrochemotherapy” (Orlowski, Belehradek, Paoletti, & Mir, 1988). Electrochemotherapy is a regional tumor treatment procedure that uses pulse electric field to increase the uptake of non-permeant, cytotoxic drugs (Lluis M. Mir et al., 2006), (Edhemovic et al., 2011). The increase of up to three orders of magnitude was observed for bleomycin after the applications of pulse electric fields (Lluis M. Mir & Orlowski, 1999), (Jaroszeski et al., 2000). In addition, reversible electroporation is used in DNA vaccination to increase intracellular delivery of vaccine plasmid (van Drunen Littel-van den Hurk & Hannaman, 2010), (Vasan et al., 2011). Animal studies show that reversible electroporation increases by two orders of magnitude the expression levels of the DNA vaccines (Drabick, Glasspool-Malone, Somiari, King, & Malone, 2001). Moreover, NIH reports on more than 17 clinical studies on the use of electroporation for mediated DNA vaccination (NIH, 2011). While reversible electroporation is very successful in the designated applications, reports show that muscle contraction and pain is an undesirable side effect of reversible electroporation (D Miklavcic et al., 2005), (Roos, Eriksson, Walters, Pisa, & King, 2009), (El-Kamary et al., 2011).

Recently, non-thermal irreversible electroporation (NTIRE) emerged as technique for soft tissue ablation (Lee, That, & Kee, 2010; B Rubinsky, 2010). The target cells are inactivated by an application of certain high strength, short duration pulse electric field (R. D Davalos, L. M Mir, & B Rubinsky, 2005; Edd, Horowitz, Davalos, Mir, & Rubinsky, 2006). The exact molecular mechanism that underlines cell death due to NTIRE treatment is not known. However, it is proposed that irreversible cell membranes' permeabilization occurs and consequently leads to cell death—(B. Rubinsky, 2007). The use of NTIRE is a minimally-invasive molecular-selective surgical technique, where electrodes in contact with the target tissue deliver electric pulses for NTIRE induction (B. Rubinsky, 2007). The important distinguished property of NTIRE tissue ablation is that other cells' structures, such as blood vessels scaffold and nerves, remain intact and neighboring cells are not affected (Lee et al., 2010; Onik, Mikus, & Rubinsky, 2007; B. Rubinsky, Onik, & Mikus, 2007). Successful treatments of prostate, liver, lung, kidney, breast and brain tumors were performed (Garcia, Pancotto et al., 2011; Neal et al., 2010; Onik & Rubinsky, 2010; Thomson, 2010).

Despite its advantage in ablation only the target tissue while preserving the tissue structure scaffold, side effects and limitations of NTIRE were reported (Ball, Thomson, & Kavnoudias, 2010; Lee et al., 2010; Thomson, 2010). The most severe of them are arrhythmias and involuntary muscle twitches (B all et al., 2010; Lee et al., 2010; Thomson, 2010). The solution to arrhythmias through a synchronizer device is described in (Ball et al., 2010). Although the strong paralytics such as cisatracurium or rocuronium (Ball et al., 2010; Lee et al., 2010) and deep anesthesia are used in clinical NTIRE treatments, muscle contractions are still observed in the proximity to the electrodes; moreover, diaphragm contractions still take place (Ball et al., 2010).

The engineering approach to medical treatment planning includes treatment design and control. A computer aided treatment design is a practical tool for surgical procedure planning In the field of electroporation based procedures the treatment planning methods optimize the electric field shape in order to ablate the undesirable tumor volume (Golberg & Rubinsky, 2010; D Miklavcic, Corovic, Pucihar, & Payselj, 2006; D Miklavcic et al., 2010; Sersa et al., 2008; Spugnini, Citro, & Porrello, 2005). In addition, significant efforts are made on heat transfer analyses to avoid heat damage (Garcia, Rossmeisl, Neal, Ellis, & Davalos, 2011; Payselj & Miklavcic, 2011). Although the muscle contraction phenomena is constantly reported in NTIRE and other electroporation applications it is missing from currently used treatment planning and theoretical analyses of electroporation based therapies. Limited attention is paid to the low strength electric field distribution around the electroporation treated areas. These, however, are sufficient to induce muscle contractions.

### SUMMARY OF THE INVENTION

An aspect of the invention is a method of reducing the non-targeted tissue volume subjected to muscular contraction during a process of electroporation of the tissue. The method comprises inserting a primary electrode into tissue to be subjected to electroporation wherein the primary electrode is inserted into the tissue to a depth “D”. A plurality of secondary electrodes are then inserted into the same tissue around the primary electrode wherein the secondary electrodes are inserted into the tissue to a depth which is more than “D” such as 10% or more, 25% or more, 50% or more, 75% or more, 100% or more, 200% or more, etc. or any percentage amounts between and above these numbers in order to create a current cage around the tissue to be subject to electroporation and thereby reduce the volume of tissue subjected to muscular contraction.

The invention includes an electrode system or configuration which is used for non-thermal electroporation comprised of a first electrode and a plurality of second electrodes positioned around the first electrode wherein the first electrode has a charge opposite that of the second electrodes and further wherein the first electrode has a length dimension 50% or more, 75% or more, 100% or more, 200% or more shorter than the length dimension of the secondary electrodes.

In another embodiment of the invention the electrode configuration is such that there are 4 or more, 8 or more, 16 or more, 20 or more secondary electrodes and the electrodes are positioned in a substantially circular pattern around the first electrode and are also positioned at substantially equal distances relative to one another.

Yet another aspect of the invention is a method of carrying out nontissue electroporation by positioning electrodes of particular sizes, shapes and configurations so as to minimize the volume of non-targeted tissue undergoing muscular contraction relative to the volume of tissue being subjected to electroporation.

Another aspect of the invention is to provide a method of carrying out tissue electroporation in a manner which minimizes muscle contractions of surrounding tissue relative to conventional systems and which reduces or eliminates the need for drugs generally used in connection with conventional electroporation systems.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 includes computer generated images 1A and 1B with FIG. 1A showing a Faradic model developed for an electroporation procedure and FIG. 1B showing a mesh configuration modeled as a closed loop.

FIG. 2 includes images 2A and 2B wherein tissue is modeled as a homogenous cylinder and FIG. 2B shows the constructed mesh.

FIG. 3 includes images 3A and 3B and can be further understood with reference to Table 3.

FIG. 4 includes images 4A and 4B where 4A includes eight electrodes and 4B includes 25 electrodes configured around a cylindrical space.

FIG. 5 includes graphs 5A and 5B which show results of the analysis of NTIRE electroporation induced electric fields in a two electrode system.

FIG. 6 includes graphs 6A, 6B, 6C, 6D, 6E and 6F showing the effect of the current cage radius, number of external electrodes (N) and penetration depth (D) of the central electrode on the volume of tissue subjected to non-thermal irreversible electroporation and the volume of tissue subjected to muscular contraction.

FIG. 7 includes images 7A, 7B and 7C showing different electrode configurations which when viewed with the data generated show designs which decrease the volume of tissue subjected to muscular contraction relative to the volume of tissue subjected to non-thermal irreversible electroporation.

### DETAILED DESCRIPTION OF THE INVENTION

Before the present electrode system and method are described, it is to be understood that this invention is not limited to particular system or method described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an electrode” includes a plurality of such electrodes and reference to “the pulse” includes reference to one or more pulses and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The invention includes the analyses of a tissue which is not an electroporation target but is still exposed to electric fields sufficient to induce muscle contraction. Mathematical models are used to characterize muscle contraction electric fields distributions and reduce muscle contractions. The invention provides specific electrode positioning design leads to electric fields shapes that reduce the volumes of a tissue exposed to fields above muscle activation threshold.

Muscles are excitable tissues, therefore they respond to the electric stimulation. Electric stimulation of excitable tissue for therapeutic purposes has a 2000 years old history (McNeal, 1977). The result of quantitative landmark work of L. Galvani (Galvani, 1791) was the fact that biological tissue responds to the externally applied electric fields. Currently, two types of tissue stimulation are recognized: direct (Voltaic) and interrupted (Faradic) stimulation (Reilly, 1998). During the Voltaic stimulation the current is directly injected into the tissue. In contrast, in Faradic stimulation the tissue excitation is achieved by exposing it to time varying electromagnetic fields (Reilly, 1998).

The application of external electric fields to muscle tissue can lead to several phenomena, which are a function of a stimulation strength and frequency. For instance, sensation, muscle contraction, cardiac reaction, thermal effect, reversible and irreversible electroporation may occur (Reilly, 1998). The threshold for each of these events vary as a function of personal tolerance, tissue electrical properties, characteristics of electric fields and electrode configuration (Reilly, 1998; Seireg & Arada-Moreno, 1981). The electric field parameters of amplitude, frequency and phase of electric currents define the threshold (Reilly, 1998; Sten-Knudsen, 1954). It was shown that a muscle twitch is the result of three mechanisms. First, a twitch results from a multiple involuntary spinal reflex response (through peripheral nerves or primarily motor nerves). Second, contractions may result from direct motor-neuron electrical stimulation in the region of electrode contact (Despa et al., 2009). Finally, the contractions may result also from a direct stimulation of denerved muscles (D Miklavcic et al., 2005).

The majority of research on muscle contraction by electric fields was done in the areas of rehabilitation (Vrbova, Hudlicka, & Centofanti, 2008) and defibrillation (Dosdall, Fast, & E. Ideker, 2010; Pumir, Romey, & Krinsky, 1998; Roth & Krassowska, 1998). Recent works on electric fields induced muscle contraction deal with DNA vaccination (Roos et al., 2009), electrochemotherapy (D Miklavcic et al., 2005; Yang, Li, Sun, Zheng, & Hu, 2009) and electronic stun devices (Despa et al., 2009; Joshi, Mishra, Song, Pakhomov, & Schoenbach, 2008; Joshi, Mishra, Xiao, & Pakhomov, 2010; Pakhomov et al., 2006). Strength-duration curves of muscle contraction for various pulse duration show that pulse duration shortening from 103 μs to 10-3 μs increased the electric field strength threshold for muscle activation from 5 V/cm to 5*104 V/cm (Rogers et al., 2004). Miklavcic et al found that increasing the frequency of pulse delivery to 5000 Hz led to a tetanus contraction of a rat's muscle during electrochemotherapy treatments (D Miklavcic et al., 2005). Even though at 5000 Hz the total force developed by muscle was twice higher than a single contraction, only a single muscle twitch took place during the delivery of 8 pulses (D Miklavcic et al., 2005). On contrary, the delivery of 8 pulses at a low frequency caused to 8 separate muscle contractions (D Miklavcic et al., 2005). DNA vaccination optimization experiment revealed that the increase of pulse delivery frequency and shortening the pulse duration, in combination with topical anesthesia, emla cream, decreased the strength of muscle twitches and increased the tolerance to the vaccination procedure (Roos et al., 2009). The problem of the electromagnetic fields effect on the non-targeted tissue was addressed in the defibrillation research, where very high fields are applied on the heart. The sock-type electrodes were proposed. These specific to heart electrode design minimized the exposure of the heart surrounding tissues to the high electric fields applied on the heart (Jayam et al., 2005; Jayanti, Zviman, S, Halperin, & Berger, 2007).

A theoretical analysis of three dimensional electric fields distribution during typical electroporation procedures are shown here. However, here we focused the analysis on characterizing electric fields strengths that are relevant to denerved muscle activation threshold [36]. We analyzed Voltaic and Faradic effects of typical clinical electroporation electric pulse sequences. The analysis shows that certain electrode configurations may significantly reduce the volumes of tissues that while not the targets of electroporation, are nevertheless exposed to electric fields strong enough to induce muscle contraction. Specifically, we find that a certain configuration of electrode arrays, which we named Current Cage, significantly reduces the volume of tissue exposed to electric fields sufficient to initiate muscle twitches. We demonstrate the potential Current Cage advantage in the skin electroporation application.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### 2. Materials and Methods.

When muscle tissue is exposed to electric fields, sensation, muscle contraction, thermal effect, reversible and irreversible electroporation may take place depending on field strength and time of exposure to the field (Reilly, 1998). The threshold for muscle contraction is in the range of 5 V/cm (Sten-Knudsen, 1954), while the threshold for irreversible electroporation is more than two orders of magnitude higher (D. Miklavcic, Semrov, Mekid, & Mir, 2000). Therefore, without the usage of blocking agents the muscle twitch is an inevitable companion to any electroporaton procedure. Therefore, in treatment planning for electroporation it is important to know both, the volume of tissue that is subjected to electroporation inducing electric fields (Vep) as well as the volume of tissue are will experience electric fields above the muscle contraction threshold (V_{MC}). In this study, we first perform Faradic and Voltaic analyses of the Vep and V_{MC} in the framework of currently used electroporation electrode configurations. In addition, we introduce a different electrode design, a current cage, which appears to have the potential to significantly reduce V_{MC} while keeping Vep similar to that of currently used electrode design, Numerical analyses and simulations were performed using finite element method (FEM) implemented in COMSOL Multiphysics (Version 3.5a, Comsol, Sweden) and MATLAB (Version 7.1a, Mathworks, USA) software. FEM is commonly used for optimization of electroporation pulse parameters and electrode configuration (Golberg & Rubinsky, 2010; D Miklavcic & Pav\check{s}elj, 2011; D Miklavcic et al., 2010; N Payselj & D Miklavcic, 2008).

### 2.1 Theoretical Aspects of a Faradic Analysis.

The inductive effects of time varying electromagnetic fields on muscles and nerves were previously investigated and are reviewed in (Reilly, 1998). The goal of this study was to test if currently applied pulse trains through electroporation electrodes may cause inductive electric fields sufficient to induce muscle contractions. We applied time-dependent Maxwell-Ampere law equations as described in Equations (1) to (3):

$$\begin{matrix}σ \frac{\partial A}{\partial t} + ∇ X ( {μ}_{0}^{- 1} {μ}_{r}^{- 1} B ) = {J}_{e} & ( 1 ) \\ B = ∇ X A & ( 2 )\end{matrix}$$

followed by a Faraday equation (Equation 3) to get the local electric field:

$$∇ XE = - \frac{\partial B}{\partial t} \qquad ( 3 )$$

Where σ [S/M] is the local conductivity, A [V·s·m⁻¹] is a magnetic vector potential, t[sec] is the time, Jₑ [A/m²] is the externally generated current density, B [V·s·m⁻²] is the magnetic flux density, E[V/m] is the electric fields intensity μr is a relative permeability and μ0 is the permittivity of vacuum. Equations 1-3 allow us to calculate the local electric fields in the tissue when the external current flows in the closed loop.

To determine the electric potential in the analyzed region Equations (1-3) are solved subject to the external current density which are in this case of a closed loop are:

Jₑ(x,y,z)=J(x,y,z) (4)

Where x,y,z are the directions of current flow in the closed loop. Boundary conditions of the external (air domain) are handled in a standard way as magnetic insulators (Equation 5).

nXA=0 (5)

### 2.2 Theoretical Aspects of a Voltaic Analysis.

In current electrochemotherapy and NTIRE electroporation procedures the pulse lengths are significantly longer than the cell membrane charging time, which is about 1 μsec (Weaver, 2000); thus, a steady state DC analyses can be implemented in order to study electric field spatial distribution during the pulse application. In this part of the study in order to get the values of local electric fields we used Laplace steady state equation (Equation 6), separating it from transients, which we took into consideration in section 2.1.

∇(σ(φ)=0 (6)

where, σ [S/M] is the local conductivity and φ[V] is the local potential.

To determine the electrical potential in the analyzed region Equation (6) is solved subject to the boundary condition which are:

φ(Σ₁)=V₀(7)

φ(Σ₂)=0 (8)

where Σ₁:Σ₂ are the geometrical locations of the electroporation electrode boundaries.

Boundary conditions that do not relate to the electrodes are handled as electrical insulating boundaries (equation 4).

n*J=0 (9)

Where J is an electrical current density (A/cm²).

The solution to Equations (6) to (9) yields to the electric field distribution in the treated tissue. The postprocessing integration calculates the volumes of tissue which are exposed to the electric fields of interest. The goal of the treatment planning optimization process will be to maximize the Vₑₚ and at the same time reduce the V_{MC}. V_{E} was defined in this model as a V[cm³] of tissue in which E>800 V/cm. V_{MC} was defined in this model as a V[cm³] of tissue in which E>5 V/cm. It should be emphasized that the Vₑₚ value is not necessarily the optimal design, because the shape of the electroporated volume relative to the targeted tissue is of key interest. However, in this study we will focus only on the relationship between the volumes of electroporation and of muscle contraction as a first step towards V_{MC} cognizant treatment planning. Furthermore, to focus ideas we will deal in this study with the volumes of NTIRE. Nevertheless, the same concepts of analysis are also relevant to reversible electroporation induced DNA vaccination and electrochemotherapy.

### 3 Numerical Model.

### 3.1 Faradic Analysis.

3.1.1 Geometry and meshing

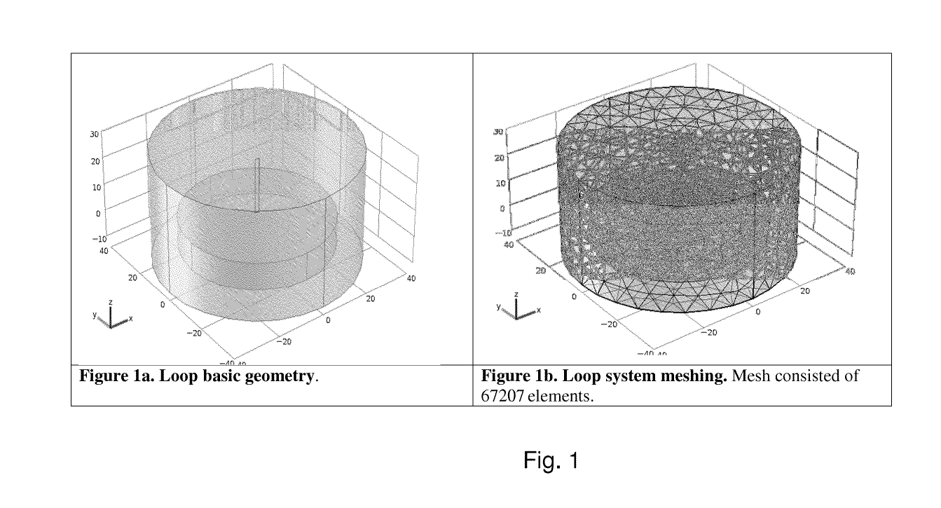

The Faradic model developed in this work is inspired by the typical percutaneous NTIRE procedure, illustrated in FIG. 1 a. The inductive component of the procedure is modeled as the closed loop in FIG. 1b.

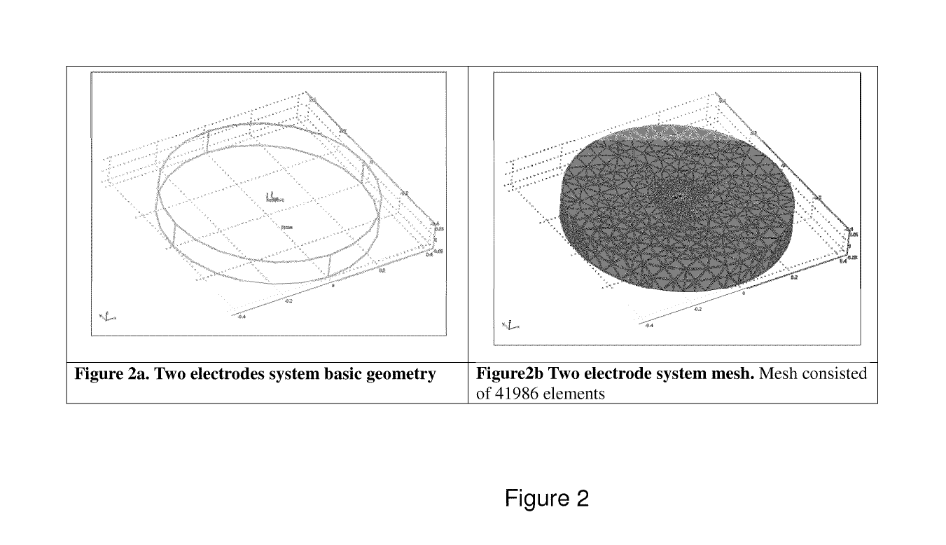

The computer model developed to simulate the inductive component of the electroporation procedure in FIGS. 1 a and 1 b is shown in FIG. 2. In this part of the analysis tissue was modeled as a homogeneous cylinder. The electrodes were modeled as cylinders composing an external loop (FIGS. 1b and 2a). The constructed mesh appears in FIG. 2b. The geometry and the mesh parameters used in the model appear in Table 1.

TABLE 1
Faradic analysis. Model geometry and mesh

| Model Part | Geometry | Mesh |
|---|---|---|
| Air | Cylinder with 40 cm | Tetrahedral elements. In order to increase the |
| | radius and 40 cm height. | resolution we added an additional domain |
| | | (cylinder 15 × 1 cm) on the border between the |
| | | loop and the tissue. Tetrahedral elements were |
| | | used. In the large air domain element size was |
| | | 1.4-8 cm while in the narrow region 0.32-4 cm |
| | | elements were used. |
| Tissue | Cylinder with 30 cm | Tetrahedral elements. In order to increase the |
| | radius and 10 cm height. | resolution we added and an additional domain |
| | | (cylinder 15 × 1 cm) in the border between the |
| | | loop and the tissue. Tetrahedral elements were |
| | | used. In the large tissue domain element size |
| | | was 1.4-8 cm while in the marrow region |
| | | 0.32-4 cm elements were used. |
| Loop | 4 Cylinders with 0.4 mm | Tetrahedral elements. Element size: 1.4-8 cm |
| | radius. 2 cylinders of | |
| | 20 cm length (on left | |
| | and right sides of the | |
| | loop) and two with | |
| | variable length (h) (i.e. | |
| | distance between the | |
| | vertical electrodes) (top | |
| | and bottom of the loop) | |

3.1.2 External Current Density and Solution.

We simulated the current, flowing through the external loop, as a smoothed rectangular pulse wave. The parameters for the current were adopted from (Bertacchini et al., 2007). The pulse used in this study was of 100 μs duration with 20 μs pause between the pulses and 2 μs transition zones (pulse upraise and decay time). The amplitude of the current was 400 A/mm²(Bertacchini et al., 2007). Tissue conductivity was assumed to be 0.33 [S/m] and relative permittivity was 40496 (Andreuccetii, Fossi, & Petrucci). The air conductivity was assumed to be 0.01 to simplify the computation process. The solution was performed using COMSOL time dependent solver. The duration of the pulse wave was 1 ms. We investigated the impact of the distance between electrode (h) in the range of from 2 to 16 cm on the induced electric field. The Vₑₚ and V_{MC} were calculated.

### 3.2 Voltaic Analysis.

We performed a basic analysis on two systems. First, we analysed a standard system which is composed of two electrodes, currently used for NTIRE application (NanoKnife® System, http://www.angiodynamics.com/products/nanoknife). Second, we investigated a configuration, we refer to as Current Cage.

3.2.1 Two electrode system.

3.2.1.1 Geometry and meshing.

In this part of the analysis tissue was modeled as a homogeneous cylinder. The electrodes were modeled as cylinders of a various length, h, completely inserted in the tissue. FIG. 2 and Table 2 describe the system geometry and meshing.

TABLE 2
Voltaic analysis. Two electrodes model. Geometry and mesh.

| Model Part | Geometry | Mesh |
|---|---|---|
| Tissue | Cylinder with 30 cm | Tetrahedral elements. |
| | radius and 10 cm | Element size: 0.32-4 cm |
| | height. | |
| Electrodes | Cylinders with 0.4 mm | Tetrahedral elements. |
| | radius of variable length | Element size: 0.32-4 cm |
| | (h). | |

3.2.1.2 Boundary Conditions and Solution.

In this model we used a static analysis. A potential of 3000 Volt was applied on one electrode, while the second electrode was grounded. Tissue conductivity was assumed to be 0.2 [S/m]. The depth of penetration of both electrodes (the value of h) was changed from 1 to 5 cm and the Vₑₚ and V_{MC} were calculated.

### 3.2.2 Current Cage Analysis.

We then investigated a electrode configuration, we refer to as Current Cage. At this specific configuration the positive electrode is located in the center of an electrode array. An important and distinguishing aspect of this study is that the penetration of the central electrode was varied relative to the surrounding electrodes arrays.

3.2.2.1 Geometry and Meshing.

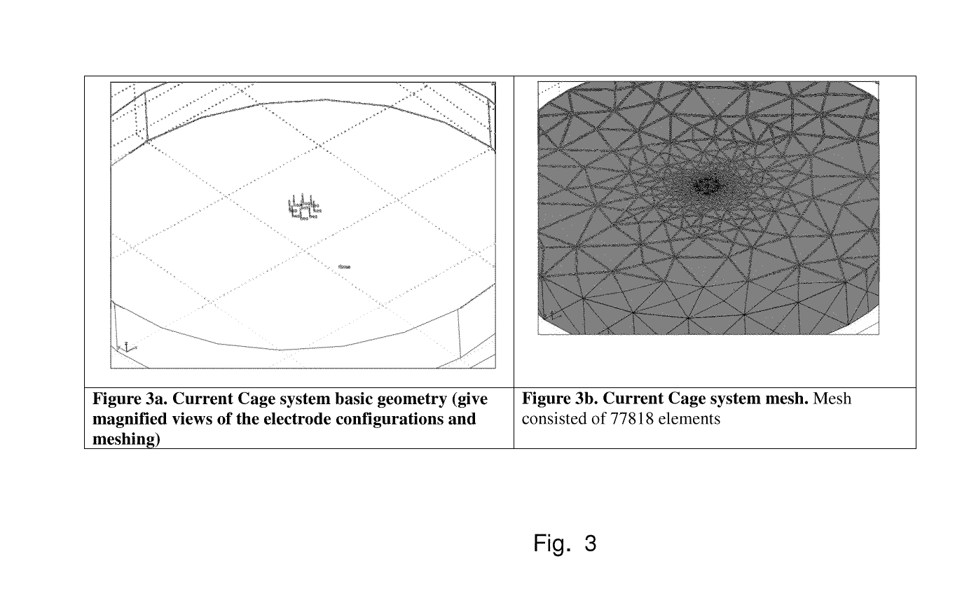

The tissue was modeled as a homogeneous cylinder. The Current Cage consists of two elements. The first element is the surrounding electrodes, whose length in this analysis was taken to be constant. The second element is the central, positive electrode which has a variable length of penetration, h. FIG. 3 and Table 3 describe the system geometry and meshing

TABLE 3
Voltaic Analysis Current Cage. Geometry and Meshing

| Model Part | Geometry | Mesh |
|---|---|---|
| Tissue | Cylinder with 30 cm | Tetrahedral elements. |
| | radius and 10 cm height. | Element size: 0.32-4 cm |
| Current Cage | Cylinders with 0.4 mm | Tetrahedral elements. |
| Surrounding | radius and 4 cm length | Element size: 0.32-4 cm |
| electrodes | | |
| Current Cage | Cylinders with 0.4 mm | Tetrahedral elements. |
| Surrounding | radius variable length (h). | Element size: 0.32-4 cm |
| electrodes | | |

3.2.2.2 Boundary Conditions and Solution.

For this model we used a static electric field COMSOL solver. A potential of 3000 Volt was applied on the central electrode, while the second electrode was grounded. We tested the impact of the Current Cage radius, number of grounded electrodes and the central electrode penetration depth on Vep and V_{MC}. We tested cage radiuses of 1, 1.5 and 2 cm. The number of electrodes in the cage varied from 2 to 64. The penetration depth of the cage was 4 cm and was kept constant, while the penetration depth of the central electrode (h) varied from 1 to 5 cm. The tissue conductivity was assumed to be 0.2 [S/m].

### 3.3 Current Cage Analysis for Skin Electroporation Application.

An important application of pulsed electric fields in medicine is for skin electroporation (Denet, Vanbever, & Preat, 2004; Gothelf & Gehl, 2010). In a typical design, pulses are applied through a two lines electrode array (4-10 electrodes in a row). Another design which resembles in structure the Current Cage design, albeit with identical length electrodes in the center and the surrounding electrode. Here we show the analysis of the two lines electrode array. In the second design the electric pulses are applied sequentially between only two electrodes from the array, and therefore the actual analysis resembles that in section 3.2.1. Significant efforts are made towards the reduction of muscle contractions in skin electroporation (Roos et al., 2009). In this section we performed a theoretical analysis that compares muscle contractions inducing conditions during skin electroporation performed through a standard 4 pins electrode array with the Current Cage design.

### 3.3.1 Geometry and Meshing

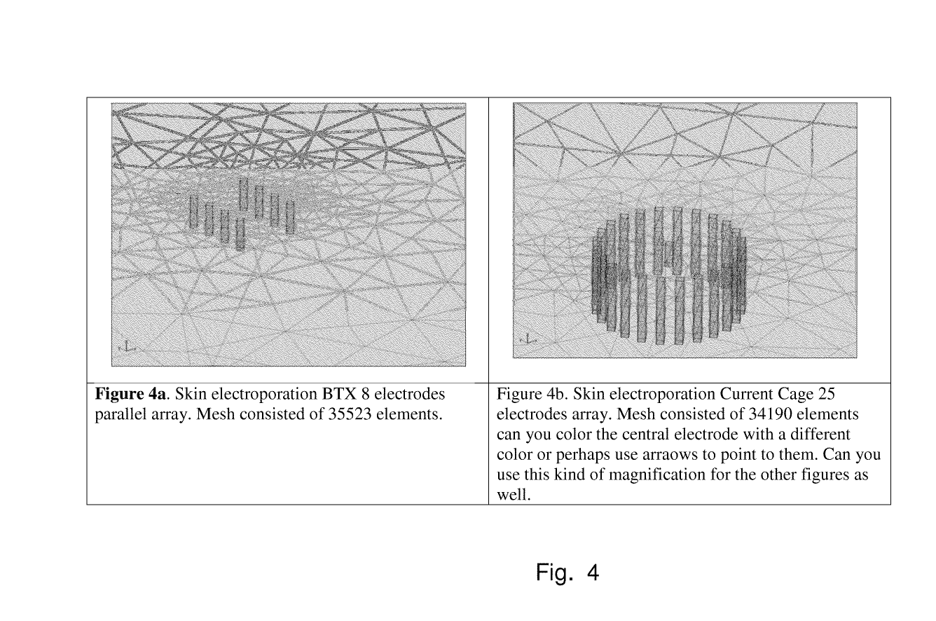

We compared a standard skin electroporation system (Harvard Apparatus BTX Parallel—Needles) 8 electrodes electrode array (FIG. 4a and Table 4) with a 25 electrodes Current Cage configuration (FIG. 4b and Table 4). In addition, we analyzed a 17 electrodes Current Cage configuration as described in Table 4.

TABLE 4
Skin electroporation. Parallel electrode array and Current Cage. Geometry
and Mesh

| Model Part | Geometry | Mesh |
|---|---|---|
| Tissue | Cylinder with 20 cm radius | Tetrahedral elements. |
| | and 10 cm height. | Element size: 0.32-4 cm |
| BTX 8 | Cylinders with 0.3 mm radius | Tetrahedral elements. |
| electrodes | and 2 mm height. Electrodes | Element size: 0.32-4 cm |
| parallel array. | are separated by 1.5 mm in | |
| | row. The parallel rows are | |
| | separated by 4 mm. | |
| Skin | Current Cage radius 4 mm. | Tetrahedral elements. |
| electroporation | External electrodes were | Element size: 0.32-4 cm |
| 25 electrodes | modeled as Cylinders with | |
| array | 0.3 mm radius and 2.5 mm | |
| Current Cage | length. | |
| | Central electrode was | |
| | modeled as Cylinder with | |
| | 0.3 mm radius and 1.3 mm | |
| | length. | |
| Skin | Current Cage radius 2.5 mm. | Tetrahedral elements. |
| electroporation | External electrodes were | Element size: 0.32-4 cm |
| 17 electrodes | modeled as Cylinders with | |
| array Current | 0.3 mm radius and 3 mm | |
| Cage | length. | |
| | Central electrode was | |
| | modeled as Cylinder with | |
| | 0.3 mm radius and 0.5 mm | |
| | length. | |

### 3.3.2 Boundary Conditions and Solution

For this model we used a static electric field COMSOL solver. In this part of the model we applied 450 V on 4 electrodes in the BTX 8 electrodes parallel array and 900 Volt on the central electrode in the Current Cage 25 electrodes array. The geometry of the Current Cage and the applied voltages were chosen in such a way that the electroporated volume (volume where E>600V/cm (Gothelf & Gehl, 2010)) was equal to the electroporated volume in the standard BTX 8 electrodes parallel array. The dermis tissue conductivity was assumed to be 0.2 [S/m]. 300 Volt were applied on the central electrode in the 17 electrodes Current Cage array.

### 4 Results.

### 4.1 Faradic Analysis.

We investigated how the external loop size impacts the volumes of tissues which are exposed to induced electric fields sufficient to cause muscle contraction V_{MC}. It was found that the induction is not sufficient to cause muscle contraction in any of the cases we studied.

### 4.2 Voltaic Analysis

### 4.2.1 The Two Electrode System.

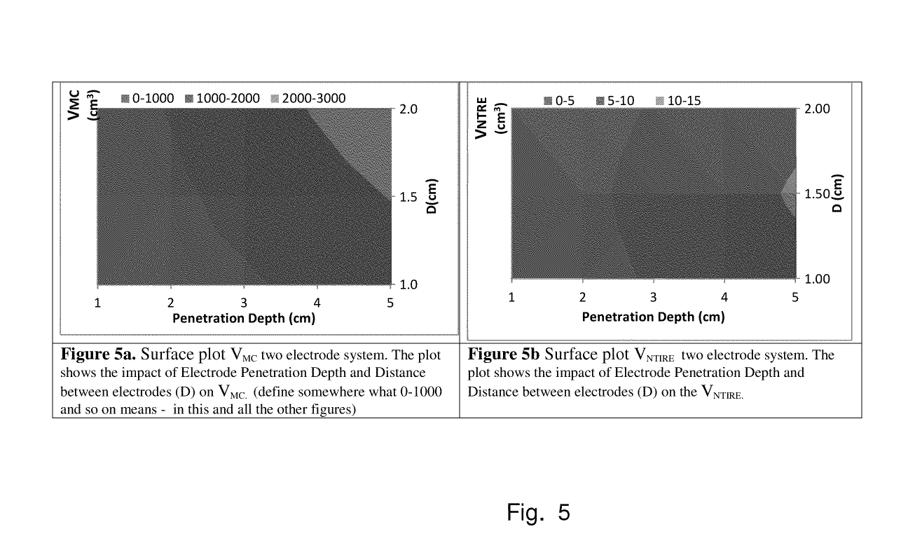

FIG. 5 shows the results of the analysis of electroporation induced electric fields in a two electrode system. FIG. 5 shows the effect of the electrode penetration depth and the distance between the two electrodes on V_{NTIRE} and V_{MC}. The simulation results imply that increasing the distance between electrodes increases the volumes of tissue that experience muscle contractions (FIG. 5a)., Interestingly, the maximum of Vep occurs when the electrodes are placed at distance of 1.5 cm from each other (FIG. 5b).

### 4.2.2 The Current Cage System.

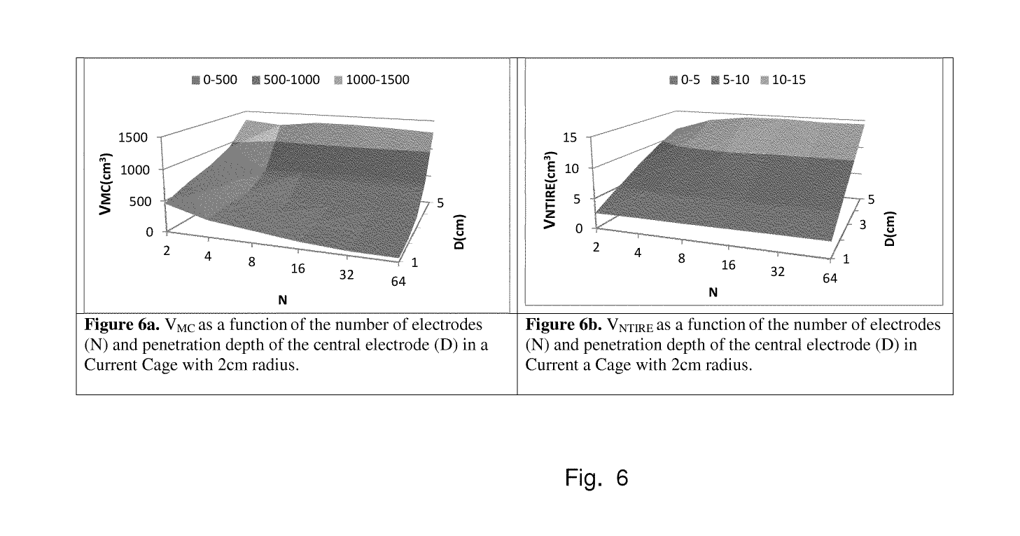

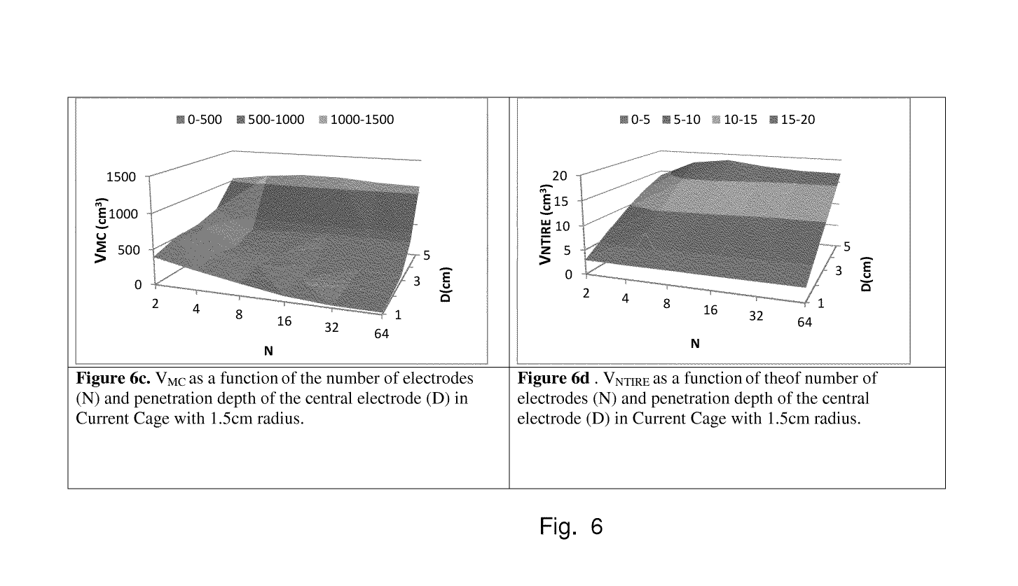

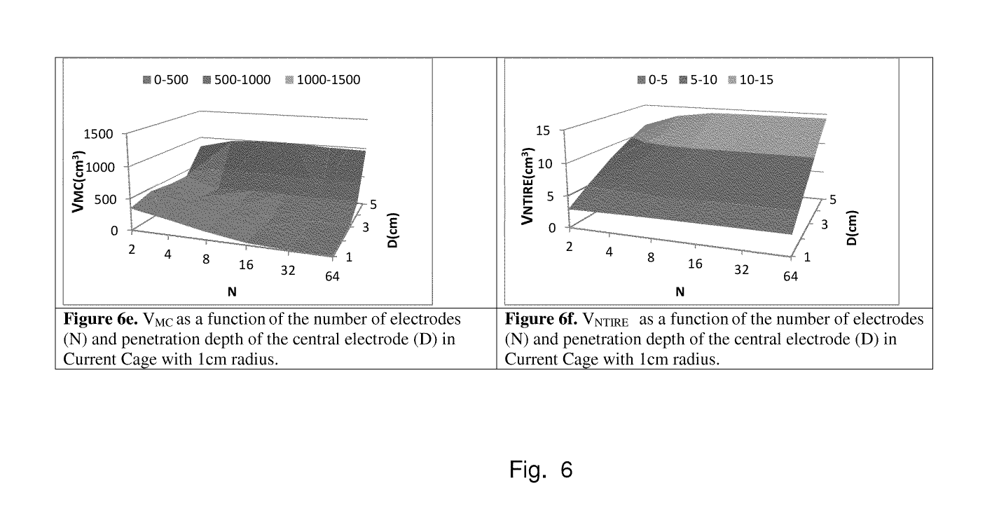

FIG. 6 shows the effect of the Current Cage radius, number of external electrodes (N) and penetration depth (D) of the central electrode on V_{NTIRE} and V_{MC}. The depth of penetration of the surrounding electrodes was kept constant. FIGS. 6a, c and e show a strong correlation between the number of electrodes used in external part of the Current Cage and V_{MC}. At the tested Current Cage radiuses of treatments (1 cm, 1.5 cm and 2 cm) N equal or greater than 16 and significantly reduces the V_{MC} (FIGS. 6b,d,f), while Vₑₚ was almost not affected by N and depended only on D. (FIGS. 6b,d,f). In addition, we show that the penetration depth (D) of the central electrode up to 3 cm does not affect V_{MC}, while Vₑₚ increases with the increase in D. Furthermore, we show that a Current Cage at radius of 1.5 cm leads to larger Vₑₚ than Cages of 1 or 2 cm radius (FIGS. 6b,d,f).

### 4.2.3 Current Cage Analysis for the Skin Electroporation Application.

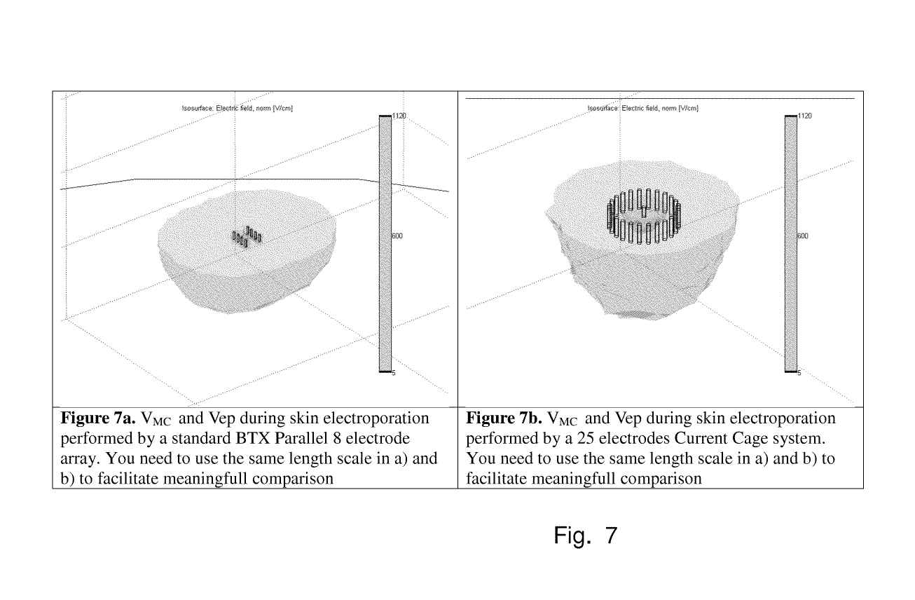

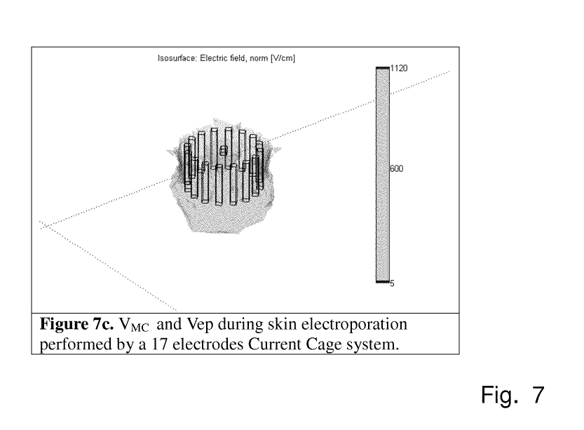

We have performed a comparison between the parallel electrode array and the Current Cage array for conditions in which the electroporation treated skin volume is the same. The reported electric field threshold for skin electropermeabilization is 600-1200V/cm (N Payselj & D Miklavcic, 2008; Payselj & Miklavcic, 2011). The V_{MC} produced as a consequence of the production of the same (Vep) is given in FIG. 7 and Table 6.

Further optimization of the Current Cage design with the goal to decrease V_{MC}/Vₑₚ led to the 17 electrodes Current Cage configuration in FIG. 7c and Table 6.

TABLE 5
Comparison of skin volumes that are exposed to threshold electric fields
during electroporation
(ad

| | | 25 electrodes | |
|---|---|---|---|
| | BTX Parallel 8 | Current | 17 electrodes Current |
| | electrode array | Cage | Cage |
| V_{MC} [mm³] | 15.09 | 2.90 | 0.147 |
| V_{ep>600 V/cm} | 0.11 | 0.11 | 0.012 |
| [mm³] | | | |
| V_{ep>1120 V/cm} | 0.04 | 0.04 | 0.005 |
| [mm³] | | | |
| V_{MC}/V_{ep>600 V/cm} | 137 | 26 | 30 |
| V_{MC}/V_{ep>1120 V/cm} | 410 | 73 | 12 |

Where:

V_{MC} [mm³]—is the volume of tissue which is exposed to E>5 V/cm.

_{Vep>600V/cm} [mm³]—is the volume of tissue, which is exposed to E>600 V/cm (Minimum threshold for tissue permeabilization).

V_{ep>1120V/cm} [mm³]—is the volume of tissue, which is exposed to E>1120 V/cm. (Full tissue permeabilisation threshold).

### 5 Discussion

The problem of muscle contraction is common to electric field based treatments such as NTIRE, electrochemotherapy, DNA vaccinations, defibrillation and electrostunning weapons (Dosdall et al., 2010; Heller, Gilbert, & Jaroszeski, 1999; Joshi, 2004; L. M Mir et al., 1998; Roos et al., 2009; Zupanic, Ribaric, & Miklavcic, 2007). The general anesthesia procedure, which is currently used to overcome this problem is labor intensive and possesses unfavorable side effects. For instance, general anesthesia suppresses the normal throat reflexes that prevent aspiration, such as swallowing, coughing, or gagging (Euliano & J. S, 2005).

Muscles fibers specialize in the transformation of electro-chemical energy into force (Rayment et al., 1993). The fiber biological membrane, serve as sensor for external triggers through the sustained transmembrane potential (Rassier, 2010). Disturbing the transmembrane potential can, under certain condition, lead to action potential and muscle contraction (Pumir et al., 1998). Extracellular electric field stimulation result in both action potential stimulation and ion channel blockage (Joshi et al., 2008; Pakhomov et al., 2006; Pumir et al., 1998). Computer simulations for spatial field distribution and excitation of nerves (Krastev & Tracey, 2009; Martinek, Stickler, Reichel, Mayr, & Rattay, 2008; Martinek, Stickler, Reichel, & Rattay, 2008), neuromuscular junction (Long, Plescia, & Shires, 2008) and denerved muscles (Reichel, Mayr, & Rattay, 1999) were reported in the past. Physical methods for muscle contraction relaxation include application of various electric fields. Ultra short high frequency (ns) pulsed electric fields were shown to be able to arrest action potential propagation (Despa et al., 2009; Joshi et al., 2008; Joshi et al., 2010; Pakhomov et al., 2006). Pre-pulses of injected current, just before the main pulse delivery, cause to the reduction of muscle contraction, probably by partially depolarization of the membrane (Horowicz & Schneider, 1981). Moreover, the ability of special type electric fields, Limoge, composed of both high and low frequency components, to induce anesthesia was proposed in the 1970's (Limoge, 1975; Limoge & Dixmerias-Iskandar, 2005). Recently, it was proposed that cell sensitization to electric stimulation may be used to reduce muscle contractions (Pakhomova et al., 2011).

Pre treatment planning for emerging electric pulses based treatment techniques such NTIRE, electrochemotherapy DNA vaccinations is an important area of research. Muscle contractions are inevitable during electroporation based treatment since the threshold for muscle activation is 2 orders of magnitude lower than the threshold for tissue electro permeabilisation. Therefore, the volumes of tissue which experience electric fields inducing muscle contractions are larger than the volumes of tissue which are treated with the various electric pulse based treatments. The invention is useful in reducing the volumes of tissue exposed to muscle contraction electric fields.

To this end, we first, performed an analysis of the effect of an external transient current loop on muscle contractions (Faradic analyses). Simulation analysis (Table 5) shows that currently used pulse electric field protocols do not induce electric fields that can cause muscle contraction. These results are in agreement with previous studies that investigated the thresholds for nerve and muscle stimulation during the application of variable magnetic fields (Reilly, 1998). The simulation shows, however, that increasing the distance between the electrodes (the width of the loop) increases the penetration of induced electric fields inside the tissue. Therefore, it is possible that nanosecond pulses involving very high electric fields may cause induction induced muscle contraction.

Second, we investigated the effects of electrode configuration on V_{MC} and Vₑₚ performing Voltaic analyses. FIG. 5a for two electrodes shows that increasing penetration depth and distance between two parallel needle electrodes increases the V_{MC}. Increasing the penetration depth also increases the Vₑₚ. Although the increase of the distance between two electrodes from 1 cm to 1.5 cm led to the increase of Vₑₚ, the further increase of the distance from 1.5 cm to 2 cm caused to the reduction of Vₑₚ. This result is consistent with (R. D Davalos, L. M Mir, & B Rubinsky, 2005), but it is shown here in a different type of data display. This mode of display indicates that there may be an optimal electrode configuration for maximal tissue ablation, and that the behavior is not monotonic. It is also evident from the results that the volume of excited muscle tissue during typical electroporation procedures is substantial.

Next we investigated the spatial distribution of electric fields in the proposed Current Cage design. The idea behind this design is based on the fact that in electroporation based treatment there is the need to produce electroporative electric fields strengths in a very specific volume (Vₑₚ), while minimizing V_{MC}. The concept of a current cage was implemented for defibrillation procedures through sock-type electrodes (Jayam et al., 2005; Jayanti et al., 2007). Although the sock-type electrode configuration was successfully implemented for the heart; the heart is a separate organ, relatively easy for de-entangling. In contrast, in electroporation based treatments the electrodes are usually inserted into the tissue. To our knowledge this is the first work which proposes usage of current cage type electrodes for decreasing the exposure of non-target tissues surrounding the electroporation treatment volumes to muscle contractions inducing electric fields.

FIG. 6 shows the effect of the number of grounded electrodes in the external cage (N) and the penetration depth of the central electrode (D) on V_{MC} and Vₑₚ, in Current Cages of 1, 1.5 and 2 cm radius. FIG. 6 shows that different from two electrodes configuration (FIG. 5) a current cage design may reduce V_{MC} while keeping the same Vₑₚ. FIGS. 6a,c,e show that an increase of N (to 16 and more) and reduction of D decrease V_{MC}. At the same time the Vₑₚ does not depend on N and increases with larger values of D (FIG. 6b,d,f). The Current Cage design of this study is different from previously proposed circle electrode arrays (Spugnini et al., 2005) or circle electrode arrays with a positive electrode in the middle (Sersa et al., 2008). In those designs all the electrodes have the same length. We find that when the central electrode penetration depth is equal or larger than the penetration depth of the surrounding grounded electrodes the V_{MC} is significantly higher compared to the configuration when the penetration depth of the central electrode is smaller than that of the surrounding electrodes.

Finally we compared the Current Cage system with the commercially available BTX Parallel 8 electrode array. We assumed the threshold of skin permeabilisation to be 600-1120 V/cm (N Payselj & D Miklavcic, 2008; Payselj & Miklavcic, 2011) and compared the V_{MC} in the two systems for the same Vₑₚ (FIGS. 7a,b and Table 6). The Voltaic analyses revealed that while the ratio of V_{MC}/V_{ep>600V/cm} and V_{MC}/V_{ep>1120Vcm} was 135 and 410 in the commercial BTX Parallel 8 electrode array, it was 73 and 26 in the 25 electrodes Current Cage design (Table 6, column 1 and 2).

The V_{MC}, V_{ep>600V/cm}, and V_{ep>1120V/cm} were the same in the both systems. Moreover, we showed that a further decrease in V_{MC}/V_{ep>600V/cm} and V_{MC}/V_{ep>1120V/cm} is possible using 17 electrodes Current Cage configuration (FIG. 7c Table 6, column 3) by decreasing the cage radius and central electrode penetration depth.

This study suggests to include minimization of V_{MC} to the electrode configuration optimization pretreatement planning. We show that certain electrode configurations reshape the electric field distribution in the tissue, exposing some tissue volumes to electric fields higher than electroporation threshold on one hand and diminishing the exposure of the nearby tissues to electric fields sufficient to induce denerved muscle contraction on another. It is important to point out that this is a primary theoretical model and future experimental studies are needed for verification.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

### REFERENCES

Andreuccetii, D., Fossi, R., & Petrucci, C. Dielectric Properties of Body Tissues:Output data. Italian natinal Research Council Institute for Applied Physics IFAC.http://niremf.ifac.cnr.it/tissprop/htmlclie/htmlclie.htm#atsfta.
Ball, C., Thomson, K., & Kavnoudias, H. (2010). Irreversible Electroporation: A New Challenge in “Out of Operating Theater” Anesthesia. Anesth Analg, 110, 1305-1309.
Bertacchini, C., Margotti, P. M., Bergamini, E., Lodi, A., Ronchetti, M., & Cadossi, R. (2007). Design of an Irreversible Electroporation System for Clinical Use. Tech Cancer Res Treat, 6(4), 313-320.
Davalos, R. D., Mir, L. M., & Rubinsky, B. (2005). Tissue ablation with Irreversible Electroporation. Annals of Biomedical Engeneering, 33(2), 223-231.
Davalos, R. D., Mir, L. M., & Rubinsky, B. (2005). Tissue ablation with Irreversible Electroporation. Annals of Biomedical Engineering, 33(2), 223-231.
Denet, A.-R., Vanbever, R., & Preat, V. (2004). Skin electroporation for transdermal and topical delivery. Advanced Drug Delivery Reviews, 56, 659-674.
Despa, F., Basati, S., Zhang, Z. D., D'Andrea, J., Reilly, J. P., Bodnar, E. N., et al. (2009). Electromuscular Incapacitation Results From Stimulation of Spinal Reflexes. Bioelectromagnetics 30, 411-421.
Dev, S. B., Rabussay, D. P., Widra, G., & Hofmann, G. A. (2000). Medical Applications of Electroporation. IEEE Transactoins on Plasma Science, 28(1), 206-223.
Dosdall, D. J., Fast, V. G., & E. Ideker, R. E. (2010). Mechanisms of Defibrillation. Annu. Rev. Biomed. Eng, 12, 233-258.
Drabick, J. J., Glasspool-Malone, J., Somiari, S., King, A., & Malone, R. W. (2001). Cutaneous Transfection and Immune Responses to Intradermal Nucleic Acid Vaccination Are Significantly Enhanced by in Vivo Electropermeabilization. Molecular Therapy, 3(2), 249-255.
Edd, J. F., Horowitz, L., Davalos, R. D., Mir, L. M., & Rubinsky, B. (2006). In vivo results of a new focal tissue ablation technique: irreversible electroporation. IEEE T Biomed Eng, 153, 1409-1415.
Edhemovic, I., Gadzijev, E. M., Brecelj, E., Miklavcic, D., Kos, B., Zupanic, A., et al. (2011). Electrochemotherapy: a New Technological Approach in Treatment of Metastases in the Liver Technology in cancer research & treatment, 475-485.
El-Kamary, S. S., Billington, M., Deitz, S., Colby, E., Rhinehart, H., Wu, Y., et al. (2011). Safety and Tolerability of the Easy Vax[trade] Clinical Epidermal Electroporation System in Healthy Adults. Molecular Therapy.
Euliano, T. Y., & J. S, G. (2005). Essential Anesthesia: From Science to Practice New York: Cambridge University Press.
Galvani, L. (1791). De viribus electricitatis in motu musculari commentarius. Bon. Sci. Art. Inst. Acad. Comm., 7, 363-418.
Garcia, P., Pancotto, T., Rossmeisl, J. J., Henao-Guerrero, N., Gustafson, N., Daniel, G., et al. (2011). Non-thermal irreversible electroporation (N-TIRE) and adjuvant fractionated radiotherapeutic multimodal therapy for intracranial malignant glioma in a canine patient. Technol Cancer Res Treat, 10(1), 73-83.
Garcia, P., Rossmeisl, J. J., Neal, R., 2nd, Ellis, T., & Davalos, R. (2011). A Parametric Study Delineating Irreversible Electroporation from Thermal Damage Based on a Minimally Invasive Intracranial Procedure. Biomedical Engineering online.
Golberg, A., & Rubinsky, B. (2010). A statistical model for multidimensional irreversible electroporation cell death in tissue. BioMedical Engineering OnLine, 9:13, doi:10.1186/1475-1925X-1189-1113.
Gothelf, A., & Gehl, J. (2010). Gene Electrotransfer to Skin; Review of Existing Literature and Clinical Perspectives. Current Gene Therapy, 10, 287-299.
Heller, R., Gilbert, R., & Jaroszeski, M. J. (1999). Clinical applications of electrochemotherapy. Adv Drug Deliv Rev, 35, 119-129.
Horowicz, P., & Schneider, M. F. (1981). Membrane charge moved at contraction thresholds in skeletal muscle fibres. J Physiol 314, 595-633.
Jaroszeski, M. J., Dang, V., Pottinger, C., Hickey, J., Gilbert, R., & Heller, R. (2000). Toxicity of anticancer agents mediated by electroporation in vitro. Anti-Cancer Drugs, 11(3), 201-208.
Jayam, V., Zviman, M., Jayanti, V., Roguin, A., Halperin, H., & Berger, R. D. (2005). Internal defibrillation with minimal skeletal muscle activation: a new paradigm toward painless defibrillation. Heart Rhythm., 2(10), 1114-1115.
Jayanti, V., Zviman, M. M., S, N., Halperin, H. R., & Berger, R. D. (2007). Novel electrode design for potentially painless internal defibrillation also allows for successful external defibrillation. J Cardiovasc Electrophysiol., 18(10), 1095-1100.
Joshi, R. P. (2004). Modeling Electrode-Based Stimulation of Muscle and Nerve by Ultrashort Electric Pulses. IEEE T Plasma Sci, 32(4), 1687-1695.
Joshi, R. P., Mishra, A., Song, J., Pakhomov, A. G., & Schoenbach, K. H. (2008). Simulation studies of ultrashort, high-intensity electric pulse induced action potential block in whole-animal nerves. IEEE Trans Biomed Engr, 55, 1391-1398.
Joshi, R. P., Mishra, A., Xiao, S., & Pakhomov, A. (2010). Model Study of Time-Dependent Muscle Response to Pulsed Electrical Stimulation. Bioelectromagnetics 31, 361-370.
Krastev, P., & Tracey, B. (2009). Modeling of Nerve Stimulation Thresholds and Their Dependence on Electrical Impedance with COMSOL. Paper presented at the Proceedings of the COMSOL Conference, Boston.
Lee, E. W., That, S., & Kee, S. T. (2010). Irreversible Electroporation: A Novel Image-Guided Cancer Therapy. Gut Liver, 4(Suppl. 1), S99-S104.
Limoge, A. (1975). An Introduction to Electroanesthesia. Baltimore: University Park Press.
Limoge, A., & Dixmerias-Iskandar, F. (2005). A Personal Experience Using Limoge's Current During a Major Surgery. Anaesthesia, 99, doi: 10.1213/1201.ANE.0000127906.0000117306.0000127901.
Long, G. L., Plescia, D., & Shires, P. K. (2008). Finite Element Analysis of Muscular Contractions from DC Pulses in the Liver. Paper presented at the Proceedings of the COMSOL Conference, Boston.
Martinek, J., Stickler, Y., Reichel, M., Mayr, W., & Rattay, F. (2008). A Novel Approach to Simulate Hodgkin-Huxley-like Excitation With COMSOL Multiphysics. Artificial Organs, 32(8), 614-619.
Martinek, J., Stickler, Y., Reichel, M., & Rattay, F. (2008). Simulating Hodgkin-Huxley-like Excitation using Comsol Multiphysics. Paper presented at the Proceedings of the COMSOL Conference, Hannover.
McNeal, N. R. (1977). 2000 years of electrical stimulation. In F. T. Hambrecht & J. B. Reswick (Eds.), Functional Electrical Stimulation:Applications in Neural Prostheses (pp. 3-35). New York: Marcel Dekker.
Miklavcic, D., Corovic, S., Pucihar, G., & Payselj, N. (2006) Importance of tumour coverage by sufficiently high local electric field for effective electrochemotherapy. EJC Supplements (4), 45-51.
Miklavcic, D., & Pav\check{s}elj, N. (2011). Resistive heating and electropermeabilization of skin tissue during in vivo electroporation: A coupled nonlinear finite element model. International Journal of Heat and Mass Transfer, 54, 2294-2302.
Miklavcic, D., Pucihar, G., Pavlovec, M., Ribaric, S., Mali, M., Macek-Lebar, A., et al. (2005). The effect of high frequency electric pulses on muscle contractions and antitumor efficiency in vivo for a potential use in clinical electrochemotherapy. Bioelectrochemistry, 65(2), 121-128.
Miklavcic, D., Semrov, D., Mekid, H., & Mir, L. M. (2000). A validated model of in vivo electric field distribution in tissues for electrochemotherapy and for DNA electrotransfer for gene therapy. Biochim. Biophys. Acta 1523 73-83.
Miklavcic, D., Snoj. M, Zupanic, A., Kos, B., Cemazar, M., Kropivnik, M., et al. (2010). Towards treatment planning and treatment of deep-seated solid tumors by electrochemotherapy. Biomedical Engineering online, 9.
Mir, L. M. (2001). Therapeutic perspectives of in vivo cell electropermeabilization. Bioelectrochemistry, 53(1), 1-10.
Mir, L. M., Gehl, J., Sersa, G., Collins, C. G., Garbay, J.-R., Billard, V., et al. (2006). Standard operating procedures of the electrochemotherapy: Instructions for the use of bleomycin or cisplatin administered either systemically or locally and electric pulses delivered by the Cliniporator™ by means of invasive or non-invasive electrodes. European Journal of Cancer, 4(11), 14-25.
Mir, L. M., Glass, L., Sersa, G., Teissié, J., Domenge, C., Miklavcic, D., et al. (1998). Effective treatment of cutaneous and subcutaneous malignant tumors by electrochemotherapy. Br J Cancer, 77(77), 2336-2342.
Mir, L. M., & Orlowski, S. p. (1999). Mechanisms of electrochemotherapy. Advanced Drug Delivery Reviews, 35(1), 107-118.
NanoKnife® System, http://www.angiodynamics.com/products/nanoknife.
Neal, R., 2nd, Singh, R., Hatcher, H., Kock, N., Torn, S., & Davalos, R. (2010). Treatment of breast cancer through the application of irreversible electroporation using a novel minimally invasive single needle electrode. Breast Cancer Res Treat, 123, 295-301.
Neumann, E., Schaefer-Ridder, M., Wang, Y., & Hofschneider, P. H. (1982). Gene transfer into mouse lyoma cells by electroporation in high electric fields. EMBO Journal, 1(7), 841-845.
NIH. (2011). Clinical trials http://clinicaltrials.gov. Accessed Dec. 4, 2011.
Okino, M., & Mohri, H. (1987). Effects of a high-voltage electrical impulse and an anticancer drug on in vivo growing tumors. Japanese Journal of Cancer Research, 78(12), 1319-1321.
Onik, G., Mikus, P., & Rubinsky, B. (2007). Irreversible electroporation: implications for prostate ablation. Technology in cancer research & treatment, 6, 295-300.
Onik, G., & Rubinsky, B. (2010). Irreversible Electroporation: First Patient Experience Focal Therapy of Prostate Cancer. In B. Rubinsky (Ed.), Irreversible Electroporation (pp. 235-247): Springer.
Orlowski, S., Belehradek, J., Jr., Paoletti, C., & Mir, L. M. (1988). Transient electropermeabilization of cells in culture. Increase of the cytotoxicity of anticancer drugs. Biochemical Pharmacology, 37(24), 4727-4733.
Pakhomov, A., Kolb, J. F., Joshi, R. P., Schoenbach, K. H., Dayton, T., Comeaux, J., et al. (2006). Neuromuscular disruption with ultrashort electrical pulses. Proc SPIE-Int Soc Opt Eng 6219, 621903-621910.
Pakhomova, 0. N., Gregory, B. W., Khorokhorina, V. A., Bowman, A. M., Xiao, S., & Pakhomov, A. (2011). Electroporation-Induced Electrosensitization. PLoS ONE, 6(2), e17100. doi:17110.11371/journal.pone.0017100.
Payselj, N., & Miklavcic, D. (2008). Numerical modeling in electroporation-based biomedical applications. Radiology and Oncology, 42, 159-168.
Payselj, N., & Miklavcic, D. (2008). Numerical Models of Skin Electropermeabilization Taking Into Account Conductivity Changes and the Presence of Local Transport Regions. IEEE Transactions on Plasma Science, 36(4), 1650-1658.
Payselj, N., & Miklavcic, D. (2011). Resistive heating and electropermeabilization of skin tissue during in vivo electroporation: A coupled nonlinear finite element model. International Journal of Heat and Mass Transfer, 54, 2294-2302.
Pumir, A., Romey, G., & Krinsky, V. (1998). Deexcitation of Cardiac Cells. Biophys J, 74, 2850-2861.
Rassier, D. E. (2010). Muscle Biophysics. From Molecules to Cells. London: Springier.
Rayment, I., Holden, H. M., Whittaker, M., Yohn, C. B., Lorenz, M., Holmes, K. C., et al. (1993). Structure of the actin-myosin complex and its implications for muscle contraction. Science, 261, 58-65.
Reichel, M., Mayr, W., & Rattay, F. (1999). Computer simulation of field distribution and excitation of denervated muscle fibers caused by surface electrodes. Artificial Organs, 23(5), 453-456.
Reilly, J. P. (1998). Applied Bioelectricity: From Electrical Stimulation to Electropathology New York: Springier.
Rogers, W. R., Merrit, J. H., Comeaux, J. A., Kuhnel, C. T., Moreland, D. F., Teltschik, D. G., et al. (2004). Strength-Duration Curve for an Electrically Excitable Tissue Extended Down to Near 1 Nanosecond. IEEE T Plasma Sci, 32(3), 1587-1599.
Roos, A.-K., Eriksson, F., Walters, D. C., Pisa, P., & King, A. D. (2009). Optimization of Skin Electroporation in Mice to Increase Tolerability of DNA Vaccine Delivery to Patients. Molecular Therapy 17(7), 1637-1642.
Roth, B. J., & Krassowska, W. (1998). The induction of reentry in cardiac tissue. The missing link: How electric fields alter transmembrane potential. Chaos, 8(1), 204-220.
Rubinsky, B. (2007). Irreversible electroporation in medicine. Tech Cancer Res Treat, 6(4), 255-260.
Rubinsky, B. (2010). Irreversible electroporation: Springer.
Rubinsky, B., Onik, G., & Mikus, P. (2007). Irreversible Electroporation: A New Ablation Modality—Clinical Implications. Technology in Cancer Research and Treatment, 6(1), 37-48.
Seireg, A., & Arada-Moreno, J. (1981). Investigation of over-skin electrical stimulation parameters for different normal muscles and subjects. J Biomech, 14(9), 587-593.
Sersa, G., Miklavcic, D., Cemazar, M., Rudolf, Z., Pucihar, G., & Snoj. M. (2008). Electrochemotherapy in treatment of tumours. EJSO, 34, 232-240.
Spugnini, E. P., Citro, G., & Porrello, A. (2005). Rational design of new electrodes for electrochemotherapy. Journal of Experimental & Clinical Cancer Research, 24(2), 246-254.
Sten-Knudsen, O. (1954). The ineffectiveness of the ‘window field’ in the initiation of muscle contraction. J Physiol, 125(2), 396-404.
Thomson, K. (2010). Human Experience with Irreversible Electroporation. In B. Rubinsky (Ed.), Irreversible Electroporation (pp. 249-254): Springer.
Titomirov, A. V., Sukharev, S., & Kistanova, E. (1991). In vivo electroporation and stable transformation of skin cells of newborn mice by plasmid DNA. Biochimica et Biophysica Acta, 1088(1), 131-134.
van Drunen Littel-van den Hurk, S., & Hannaman, D. (2010). Electroporation for DNA immunization: clinical application. Expert Review of Vaccines, 9(5), 503-517.
Vasan, S., Hurley, A., Schlesinger, S. J., Hannaman, D., Gardiner, D. F., Dugin, D. P., et al. (2011). Electroporation Enhances the Immunogenicity of an HIV-1 DNA Vaccine Candidate in Healthy Volunteers. PLoS ONE, 6(5), e19252.
Vrbova, G., Hudlicka, O., & Centofanti, K. S. (2008). Application of Muscle/nerve Stimulation in Health and Disease Amsterdam.
Weaver, J. C. (2000). Electroporation of cells and tissues. IEEE Transactions on Plasma Science, 28, 24-33.
Yang, X. J., Li, J., Sun, C. X., Zheng, F. Y., & Hu, L. N. (2009). The effect of high frequency steep pulsed electric fields on in vitro and in vivo antitumor efficiency of ovarian cancer cell line skov3 and potential use in electrochemotherapy. J Exp Clin Cancer Res., 28(1), 1-9.
Zupanic, A., Ribaric, S., & Miklavcic, D. (2007). Increasing the repetition frequency of electric pulse delivery reduces unpleasant sensations that occur in electrochemotherapy. Neoplasma 54(54), 246-250.

1. An electrode configuration used for electroporation, comprising:
a first electrode; and a plurality of secondary electrodes positioned around the first electrode;
wherein the first electrode has a charge opposite to the second electrode's charge, and
further wherein the first electrode has a length dimension 80% or less shorter than a length dimension of the secondary electrodes.

2. The electrode configuration of claim 1, wherein the first electrode length dimension is 100% or less shorter than the length dimension of the secondary electrode.

3. The electrode configuration of claim 1, wherein each of the secondary electrodes is positioned at a substantially equal distance from the first electrode.

4. The electrode configuration of claim 3, wherein there are four or more secondary electrodes.

5. The electrode configuration of claim 3 wherein there are eight or more secondary electrodes and the secondary electrodes are equally spaced apart from each other in a circle around the first electrode.

6. The electrode configuration of claim 3 wherein there are sixteen or more secondary electrodes and the secondary electrodes are equally spaced apart from each other in a circle around the first electrode.

7. The electrode configuration of claim 3 wherein there are thirty-two or more secondary electrodes and the secondary electrodes are equally spaced apart from each other in a circle around the first electrode.

8. The electrode configuration of claim 3 wherein there are sixty-four or more secondary electrodes and the secondary electrodes are equally spaced apart from each other in a circle around the first electrode.

9. The electrode configuration of claim 3 wherein there are 128 or more secondary electrodes and the secondary electrodes are equally spaced apart from each other in a circle around the first electrode.

10. The electrode configuration of claim 3 wherein there are 256 or more secondary electrodes and the secondary electrodes are equally spaced apart from each other in a circle around the first electrode.

11. A method of reducing non-target tissue volume subjected to muscular contraction during a process of electroporation, comprising:
inserting a primary electrode into tissue to be subjected to electroporation (reversible or irreversible), wherein the primary electrode is inserted to a depth D;
inserting a plurality of secondary electrodes into the tissue around the primary electrode, wherein the secondary electrodes are inserted into the tissue to a depth which is 25% or more greater than D.

12. The method of claim 9, wherein the secondary electrodes are inserted into the tissue to a depth which is 50% or more greater than D.

13. The method of claim 9, wherein the secondary electrodes are inserted into the tissue to a depth which is 100% or more greater than D.

14. The method of claim 9, wherein there are eight or more secondary electrodes positioned in a circular pattern around the primary electrode and wherein the secondary electrodes are spaced at equal distances relative to each other.

15. The method of claim 9, wherein there are sixteen or more secondary electrodes positioned in a circular pattern around the primary electrode and wherein the secondary electrodes are spaced at equal distances relative to each other.

16. The method of claim 9, wherein there are thirty-two or more secondary electrodes positioned in a circular pattern around the primary electrode and wherein the secondary electrodes are spaced at equal distances relative to each other.

17. The method of claim 9, wherein there are sixty-four or more secondary electrodes positioned in a circular pattern around the primary electrode and wherein the secondary electrodes are spaced at equal distances relative to each other.

18. The method of claim 9, wherein there are 128 or more secondary electrodes positioned in a circular pattern around the primary electrode and wherein the secondary electrodes are spaced at equal distances relative to each other.

19. The method of claim 9, wherein there are 258 or more secondary electrodes positioned in a circular pattern around the primary electrode and wherein the secondary electrodes are spaced at equal distances relative to each other.

20. The procedures for the described electrode array are: NTIRE, Electrochemotherapy, DNA electrovaccination, electrogenetherapy, ionophoreses, defibrillation, brain electro stimulation.
